(19) United States
(12) Patent Application Publication
(10) Pub. No.: US 2025/0235607 A1
(43) Pub. Date: Jul. 24, 2025

(54) INFUSION PUMP STATUS CONNECTIVITY TO NURSE CALL SYSTEM AND COMMUNICATION DEVICES

(71) Applicant: Hill-Rom Services, Inc., Batesville, IN (US)

(72) Inventors: Ryan T. Hochworter, Alpharetta, GA (US); Elizabeth H. Zechman, Willow Spring, NC (US); Katherine Marie Vignoe, Peapack, NJ (US); Linh Nguyen Altork, Apex, NC (US); Eric P. Jensen, Auburn, NY (US); Donny Patel, Danville, NH (US); Connor P. Kelleher, Wauconda, IL (US); Jonah Datz, Denver, CO (US); Jeremy P. Dixon, McHenry, IL (US); Theresa M. Warner, Denver, CO (US)

(21) Appl. No.: 18/983,762

(22) Filed: Dec. 17, 2024

Related U.S. Application Data
(60) Provisional application No. 63/622,660, filed on Jan. 19, 2024.

(51) Int. Cl. A61M 5/142; A61M 5/168; G16H 10/60

(52) U.S. Cl. CPC A61M 5/142; A61M 5/16831; G16H 10/60; A61M 2205/18; A61M 2205/3553; A61M 2205/3561; A61M 2205/3592; A61M 2205/502; A61M 2205/584

(57) ABSTRACT

A healthcare information system for use in a healthcare facility includes infusion pumps that transmit pump status data to a nurse call system having a nurse call server, a status board, and a plurality of dome lights mounted in hallways adjacent to doors of respective patient rooms. The nurse call server receives the pump status data and promulgates the pump status data for display on the status board and for display one or more of wireless mobile devices carried by caregivers. The nurse call server also causes a pump status section of a dome light to be illuminated in a manner that is dependent upon the content of the pump status data originating from one or more infusion pumps in the corresponding patient room.

The present application claims the benefit, under 35 U.S.C. § 119 (e), of U.S. Provisional Patent Application No. 63/622,660, filed Jan. 19, 2024, which is hereby incorporated by reference herein in its entirety.

### BACKGROUND

The present application relates to nurse call systems and particularly, to nurse call systems that receive alerts from medical devices. More particularly, the present disclosure relates to nurse call systems that send alert messages to wireless mobile devices carried by caregivers.

In today's hospital environments, patients and caregivers are sometimes inundated with alarms from a wide variety of medical devices. Caregivers also carry mobile devices, such as smart phones, that receive alerts and other notifications about patient care in the healthcare facility. The number of device alarms per patient per day can reach several hundred depending on the unit within a hospital, translating to thousands of device alarms on every unit of the hospital, which potentially results in tens of thousands of device alarms throughout the hospital on some days. Caregiver alerts and notifications that are presented visually to the caregiver on their mobile devices and on medical devices add to the amount of actionable information to which caregivers may need to respond.

The frequent and sometimes continuous noise from device alarms, especially from unnecessary, nuisance alarms, can lead to alarm fatigue such as desensitization, elevated stress, and clinical complacency. If not properly managed by healthcare facilities, the unintended effects of clinical alarm systems causing alarm fatigue can possibly compromise patient safety. A fatigued clinician may resort to unsafe practices such as turning down the alarm volume so that an alarm can't be heard or even removing an often-alarming device from patient use. Thus, there is an ongoing need in the healthcare environment to reduce nuisance alarms and manage the occurrence of alarms that help alleviate alarm fatigue in caregivers. Systems that enhance the ability of caregivers to proactively attend to patients and/or medical devices prior to alarms occurring, particularly audible alarms, would be welcomed by caregivers.

### SUMMARY

An apparatus, system, or method may comprise one or more of the features recited in the appended claims and/or the following features which, alone or in any combination, may comprise patentable subject matter:

According to a first aspect of the present disclosure, a healthcare information system for use in a healthcare facility having a network including wireless access points may be provided. The healthcare information system may include an infusion pump gateway, one or more infusion pumps that may be configured to transmit pump status data wirelessly to at least one of the wireless access points of the healthcare facility for forwarding to the infusion pump gateway, and an enterprise gateway that may be communicatively coupled to the infusion pump gateway and that may be configured to receive the pump status data from the infusion pump gateway. The healthcare information system may further include a plurality of wireless mobile devices that each may be carried by a respective caregiver of the healthcare facility and a nurse call system that may include a nurse call server, a status board, and a plurality of dome lights that may be mounted in one or more hallways of the healthcare facility adjacent to doors of respective patient rooms of the healthcare facility. The nurse call server may be communicatively coupled to the enterprise gateway and may be configured to receive the pump status data from the enterprise gateway. The nurse call server also may be configured to promulgate the pump status data for display on the status board and for display on at least one of the plurality of wireless mobile devices. The nurse call server also may be configured to cause a pump status section of at least one of the plurality dome lights to be illuminated in a manner that is dependent upon the content of the pump status data.

In some embodiments of the healthcare information system of the first aspect, the nurse call system may include a master nurse station computer that may be communicatively coupled to the nurse call server and that may be communicatively coupled to the status board. The pump status data may be sent to the master nurse station computer which may display the pump status data. If desired, the pump status data may be displayed on the status board in a row of a table and the row also may display any one or more of the following: a room number of the respective room in which the at least one infusion pump may be located, a patient name in HIPAA-compliant format of a patient that may be assigned to the respective room, patient risk information, bed status information of a patient bed that may be in the respective room, and names of caregivers that may be assigned to the patient or to the patient room.

Optionally, the nurse call system of the first aspect may include a graphical audio station in the respective room in which the one or more infusion pumps may be located. The master nurse station computer may be communicatively coupled to the graphical audio station. Further optionally, the plurality of wireless mobile devices may be configured to display a user input which may be displayed along with the pump status data and which may be selectable to open an audio channel between the wireless mobile device and the graphical room station. The present disclosure contemplates that the pump status data may be indicative of any one or more of the following, for example: infusing, infusing near complete, and infusing complete. Alternatively or additionally, the pump status data may include pump alarms and/or notifications other than those just listed.

If desired, the plurality of wireless mobile devices of the first aspect may be configured to display, along with the pump status data, any one or more of the following: a room number of the respective room in which the at least one infusion pump may be located, a name of a patient that may be in the respective room, a first user input to accept an alert that may be associated with the pump status data, a second user input to place an audio call to the respective room, and a third user input to re-route the alert associated with the pump status data to another caregiver. Optionally, each of the plurality of wireless mobile devices may be configured so that selection of the second user input also accepts the alert associated with the pump status data.

In some embodiments of the healthcare information system of the first aspect, the pump status section of the at least one dome light may be illuminated continuously in response to the pump status data indicating that the one or more infusion pumps may be infusing an associated patient. Optionally, the pump status section of the at least one dome light may be illuminated so as to flash at a slow rate of at most once per second, just to give one arbitrary slow flash rate, in response to the pump status data indicating that the one or more infusion pumps may be nearing completion by being within thirty minutes or less (e.g., five minutes just to give one arbitrary time example) of infusing an associated patient. Further optionally, the pump status section of the at least one dome light may be illuminated so as to flash at a fast rate of at least twice per second, just to give one arbitrary fast flash rate, in response to the pump status data indicating that the one or more infusion pumps have completed infusing the associated patient. Still further optionally, the pump status section of the at least one dome light may be illuminated in a dedicated color for pump status, such as magenta just to give one arbitrary color example, when illuminated. The color of the pump status section may be customer configurable in some embodiments, such as to match a color of a housing of the pump.

With regard to the first aspect, the present disclosure contemplates that an alarm occurring at the one or more infusion pumps in a patient room may result in the nurse call system displaying an alarm message on the status board and on one or more of the wireless mobile devices of caregivers assigned to the patient room and that acknowledgment of the alarm at the one or more infusion pumps, or rectification of a condition causing the alarm at the one or more infusion pumps, may clear the alarm from the nurse call system. Clearing the alarm may result in the alarm message no longer being displayed on the status board and no longer being displayed on the one or more of the wireless mobile devices of caregivers assigned to the patient room.

If desired, the enterprise gateway of the first aspect may be communicatively coupled to an ADT server of the healthcare facility and may receive patient-to-room association data from the ADT server. It is further contemplated that the enterprise gateway may be communicatively coupled to an EMR server of the healthcare facility and may be configured to route the pump status data to the EMR server. Optionally, the healthcare information system of the first aspect further may include a clinical data repository database and the enterprise gateway may be configured to route the pump status data to the clinical data repository database. The present disclosure contemplates that the pump status data may include a notification that a fluid bag is near empty, just to give one notification example.

In some embodiments of the healthcare information system of the first aspect, the pump status data may be communicated from the one or more infusion pumps to the infusion pump gateway according to the IHE/HL7 protocol. Alternatively or additionally, the pump status data may be communicated from the infusion pump gateway to the enterprise gateway according to the TCP/IP, HL7 2.x, PCD-04 protocol. Further alternatively or additionally, communications from the enterprise gateway to the nurse call system may be according to the TCP/IP, HL7 2.x, PCD-04 protocol and communications from the nurse call system to the enterprise gateway may be according to the TCP/IP, HL7 2.x, PCD-05 protocol. In some embodiments, an alarm manager, such as an alarm manager server or alarm manager software provided in another computer device such as the enterprise gateway or a nurse call server, is provided to handle communications between the enterprise gateway and the nurse call system.

Optionally, the healthcare information system of the first aspect further may include a communications server that may be communicatively coupled to the nurse call server and that may be configured to receive the pump status data for forwarding to at least one wireless mobile device of the plurality of wireless mobile devices. In such embodiments, communications from the nurse call server to the communications server may be according to the HL7 PCD-06 protocol. Alternatively or additionally, communications between the communications server and the at least one wireless mobile device of the plurality of wireless mobile devices may be according to the Wireless Communications Transfer Protocol (WCTP).

In some embodiments of the healthcare information system of the first aspect, the infusion pump gateway and the enterprise gateway may be communicatively coupled to an EMR server of the network of the healthcare facility. In such embodiments, the pump status data may be communicated from the infusion pump gateway to the EMR server according to the HL7 protocol and the pump status data may be communicated from the EMR server to the enterprise gateway according to the Fast Healthcare Interoperability Resources 1 (FHIR) protocol and/or the HL7 protocol.

The present disclosure further contemplates that the enterprise gateway may be communicatively coupled to an ADT server of the network of the healthcare facility and the enterprise gateway may be configured to query the ADT server for patient-to-room association data. In such embodiments, in response to receiving pump status data from the one or more infusion pumps, the enterprise gateway may be configured to append the patient-to-room association data to the pump status data for the patient treated by the corresponding one or more infusion pumps. More particularly, the present disclosure contemplates that the infusion pump gateway on the enterprise gateway (aka a device bridge) appends the patient-to-room association data to the pump status data. In some embodiments of the first aspect, the nurse call server may be communicatively coupled to the enterprise gateway via an alarm manager.

According to a second aspect of the present disclosure, a healthcare information system for use in a healthcare facility having a network may be provided. The healthcare information system of the second aspect may include one or more infusion pumps that may be configured to transmit pump status data to the network, a plurality of wireless mobile devices that each may carried by a respective caregiver of the healthcare facility, and a nurse call system that may include a nurse call server, a status board, and a plurality of dome lights that may be mounted in one or more hallways of the healthcare facility adjacent to doors of respective patient rooms of the healthcare facility. The nurse call server of the second aspect may be communicatively coupled to the network and may be configured to receive the pump status data from the network. The nurse call server also may be configured to promulgate the pump status data for display on the status board and for display on at least one of the plurality of wireless mobile devices. The nurse call server also may be configured to cause a pump status section of at least one of the plurality of dome lights to be illuminated in a manner that is dependent upon the content of the pump status data.

In some embodiments, the nurse call system of the second aspect may include a master nurse station computer that may be communicatively coupled to the nurse call server and that may be communicatively coupled to the status board. In such embodiments, the pump status data may be sent to the master nurse station computer which may control display of the pump status data on the status board. If desired, the pump status data may be displayed on the status board in a row of a table and the row also may display any one or more of the following: a room number of the respective room in which the at least one infusion pump may be located, a patient name in HIPAA-compliant format of a patient that may be assigned to the respective room, patient risk information, bed status information of a patient bed in the respective room, and names of caregivers that may be assigned to the patient or to the patient room.

Optionally, the nurse call system of the second aspect may include a graphical audio station that may be in the respective room in which the one or more infusion pumps may be located. Further optionally, the master nurse station computer may be communicatively coupled to the graphical audio station. Still further optionally, the plurality of wireless mobile devices may be configured to display a user input which may be displayed along with the pump status data and which may be selectable to open an audio channel between the wireless mobile device and the graphical room station. The present disclosure contemplates that the pump status data of the second aspect may be indicative of any one or more of the following: infusing, infusing near complete, and infusing complete. Alternatively or additionally, the pump status data may include pump alarms and/or notifications other than those just listed.

If desired, the plurality of wireless mobile devices of the second aspect may be configured to display, along with the pump status data, any one or more of the following: a room number of the respective room in which the at least one infusion pump may be located, a name of a patient that may be in the respective room, a first user input to accept an alert that may be associated with the pump status data, a second user input to place an audio call to the respective room, and a third user input to re-route the alert associated with the pump status data to another caregiver. Optionally, each of the plurality of wireless mobile devices may be configured so that selection of the second user input also accepts the alert associated with the pump status data.

In some embodiments of the healthcare information system of the second aspect, the pump status section of the at least one dome light may be illuminated continuously in response to the pump status data indicating that the one or more infusion pumps may be infusing an associated patient. Optionally, the pump status section of the at least one dome light of the second aspect may be illuminated so as to flash at a slow rate of at most once per second in response to the pump status data indicating that the one or more infusion pumps may be nearing completion by being within five minutes or less of infusing an associated patient. Further optionally, the pump status section of the at least one dome light of the second aspect may be illuminated so as to flash at a fast rate of at least twice per second in response to the pump status data indicating that the one or more infusion pumps have completed infusing the associated patient. Still further optionally, the pump status section of the at least one dome light may be illuminated in a dedicated color for pump status, such as magenta just to give one arbitrary color example, when illuminated. The color of the pump status section may be customer configurable in some embodiments, such as to match a color of a housing of the pump.

With regard to the second aspect, the present disclosure contemplates that an alarm occurring at the one or more infusion pumps in a patient room may result in the nurse call system displaying an alarm message on the status board and on one or more of the wireless mobile devices of caregivers assigned to the patient room and that acknowledgment of the alarm at the one or more infusion pumps, or rectification of a condition causing the alarm at the one or more infusion pumps, may clear the alarm from the nurse call system. Clearing the alarm may result in the alarm message no longer being displayed on the status board and no longer being displayed on the one or more of the wireless mobile devices of caregivers assigned to the patient room.

Optionally, the nurse call server of the second aspect may receive patient-to-room association data from an ADT server of the network. Further optionally, the pump status data also may be routed to an EMR server of the network. Alternatively or additionally, the pump status data also may be routed to a clinical data repository database of the network. If desired, the pump status data of the second aspect may include a notification that a fluid bag may be near empty, just to give one notification example.

According to a third aspect of the present disclosure, a method may include operating one or more infusion pumps to treat a patient, transmitting pump status data wirelessly from the one or more infusion pumps to at least one wireless access point of a network of a healthcare facility for forwarding to an infusion pump gateway, receiving the pump status data at the infusion pump gateway, transmitting the pump status data from the infusion pump gateway to an enterprise gateway, and providing a nurse call system that may include a nurse call server, a status board, and a plurality of dome lights that may be mounted in one or more hallways of the healthcare facility adjacent to doors of respective patient rooms of the healthcare facility. The method further may include transmitting the pump status data from the enterprise gateway to the nurse call server (e.g., via an alarm manager, such as an alarm manager server or alarm manager software provided in the enterprise gateway or the nurse call server), receiving the pump status data at the nurse call server, transmitting the pump status data for display on the status board and at least one of a plurality of wireless mobile devices, displaying the pump status data on the status board and on the at least one of the plurality of wireless mobile devices, and operating at least one of the plurality of dome lights such that a pump status section of the at least one of the plurality dome lights may be illuminated in a manner that is dependent upon the content of the pump status data.

In some embodiments, the method of the third aspect further may include transmitting the pump status data from the nurse call server to a master nurse station computer of the nurse call system, and controlling the display of the pump status data on the status board with the master nurse station computer. If desired, displaying the pump status data on the status board may include displaying the pump status data in a row of a table and the row may include any one or more of the following: a room number of the respective room in which the at least one infusion pump may be located, a patient name in HIPAA-compliant format of a patient that may be assigned to the respective room, patient risk information, bed status information of a patient bed that may be in the respective room, and names of caregivers that may be assigned to the patient or to the patient room.

Optionally, in the method of the third aspect, the nurse call system may include a graphical audio station in the respective room in which the one or more infusion pumps may be located. Further optionally, the master nurse station computer may be communicatively coupled to the graphical audio station, and the method of the third aspect further may include displaying on the plurality of wireless mobile devices a user input along with the pump status data. Still further, the method of third aspect may include opening an audio channel between the wireless mobile device and the graphical room station in response to selection of the user input. Also in the method of the third aspect, the pump status data may be indicative of any one or more of the following: infusing, infusing near complete, and infusing complete. Alternatively or additionally, the pump status data may include pump alarms and/or notifications other than those just listed.

If desired, the method of the third aspect further may include displaying on at least one of the plurality of wireless mobile devices, along with the pump status data, any one or more of the following: a room number of the respective room in which the at least one infusion pump may be located, a name of a patient that may be in the respective room, a first user input to accept an alert that may be associated with the pump status data, a second user input to place an audio call to the respective room, and a third user input to re-route the alert associated with the pump status data to another caregiver. Each of the plurality of wireless mobile devices of the third aspect may be configured so that selection of the second user input also may accept the alert associated with the pump status data.

In some embodiments of the method of the third aspect, operating the at least one of the plurality of dome lights may include illuminating the pump status section of the at least one dome light continuously in response to the pump status data indicating that the one or more infusion pumps may be infusing an associated patient. Optionally, operating the at least one of the plurality of dome lights of the third aspect may include illuminating the pump status section of the at least one dome light so as to flash at a slow rate of at most once per second in response to the pump status data indicating that the one or more infusion pumps may be nearing completion by being within five minutes or less of infusing an associated patient. Further optionally, operating the at least one of the plurality of dome lights of the third aspect may include illuminating the pump status section of the at least one dome light so as to flash at a fast rate of at least twice per second in response to the pump status data indicating that the one or more infusion pumps may have completed infusing the associated patient. Still further optionally, the pump status section of the at least one dome light may be illuminated in a dedicated color for pump status, such as magenta just to give one arbitrary color example, when illuminated. The color of the pump status section may be customer configurable in some embodiments, such as to match a color of a housing of the pump.

The method of the third aspect further may include displaying an alarm message on the status board and on one or more of the wireless mobile devices of caregivers assigned to a patient room in response to an alarm occurring at the one or more infusion pumps in the patient room and clearing the alarm from the nurse call system in response to acknowledgment of the alarm at the one or more infusion pumps, or rectification of a condition causing the alarm at the one or more infusion pumps. In the method of the third aspect, clearing the alarm from the nurse call system may result in the alarm message no longer being displayed on the status board and no longer being displayed on the one or more of the wireless mobile devices of caregivers assigned to the patient room.

Optionally, the enterprise gateway may be communicatively coupled to an ADT server of the healthcare facility and the method of the third aspect further may include receiving at the enterprise gateway patient-to-room association data from the ADT server. Further optionally, the enterprise gateway may be communicatively coupled to an EMR server of the healthcare facility and the method of the third aspect further may include routing the pump status data to the EMR server from the enterprise gateway. Still further optionally, the method of the third aspect further may include routing the pump status data from the enterprise gateway to a clinical data repository database. With regard to the method of the third aspect, the present disclosure contemplates that the pump status data may include a notification that a fluid bag is near empty.

In some embodiments of the method of the third aspect, transmitting the pump status data wirelessly from the one or more infusion pumps to the at least one wireless access point for forwarding to the infusion pump gateway includes transmitting the pump status data according to the IHE/HL7 protocol. Alternatively or additionally, in the method of the third aspect, transmitting the pump status data from the infusion pump gateway to the enterprise gateway comprises transmitting the pump status data according to the TCP/IP, HL7 2.x, PCD-04 protocol. Further alternatively or additionally, in the method of the third aspect, transmitting the pump status data from the enterprise gateway to the nurse call server may include transmitting the pump status data according to the TCP/IP, HL7 2.x, PCD-04 protocol and communicating messages from the nurse call system to the enterprise gateway may be according to the TCP/IP, HL7 2.x, PCD-05 protocol.

The present disclosure contemplates that the method of the third aspect further may include transmitting the pump status data from the nurse call server to a communications server and forwarding the pump status data from the communications server to at least one wireless mobile device of the plurality of wireless mobile devices. For example, the pump status data may be forwarded by the communications server of the third aspect according to the HL7 PCD-06 protocol. Alternatively or additionally, the pump status data may be forwarded by the communications server of the third aspect according to the Wireless Communications Transfer Protocol (WCTP).

In some embodiments of the method of the third aspect, the infusion pump gateway and the enterprise gateway may be communicatively coupled to an EMR server of the network of the healthcare facility. In such embodiments, the method of the third aspect further may include transmitting the pump status data from the infusion pump gateway to the EMR server according to the HL7 protocol and transmitting the pump status data from the EMR server to the enterprise gateway according to the Fast Healthcare Interoperability Resources 1 (FHIR) protocol.

The present disclosure further contemplates that the enterprise gateway may be communicatively coupled to an ADT server of the network of the healthcare facility and the method of the third aspect further may include sending a query from the enterprise gateway to the ADT server for patient-to-room association data, receiving the patient-to-room association data at the enterprise server from the ADT server, appending the patient-to-room association data to the pump status data for the patient that may be treated by the corresponding one or more infusion pumps, and transmitting the patient-to-room association data to the nurse all server along with the pump status data. In some embodiments of the third aspect, transmitting the pump status data from the enterprise gateway to the nurse call server may include transmitting the pump status data from the enterprise gateway to the nurse call server via an alarm manager.

Additional features, which alone or in combination with any other feature(s), such as those listed above, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures, in which:

FIG. 1 is a rudimentary block diagram of a healthcare information system showing a plurality of infusion pumps providing pump status data to a clinical platform of a network for eventual display on a status board of a nurse call system and a mobile wireless device carried by a caregiver, and showing a pump status section or zone of a dome light of the nurse call system being illuminated based on the pump status data;

FIG. 2 is a more detailed block diagram of the healthcare information system showing an infusion pump communicating with a pump gateway which, in turn, communicates with an enterprise gateway of a device bridge and showing a nurse call server of a nurse call system communicating with the enterprise gateway via an alarm manager and also communicating with a wireless mobile device via a communications server;

FIG. 3 is screen shot of a first screen of the status board of FIG. 1 showing a table of the status board displaying rows of information for each room of a unit of a healthcare facility and showing one of the rows indicating that the pump status data for one of the patient rooms is “Infusing;”

FIG. 4 is a screen shot of a second screen of the status board, similar to FIG. 3, but showing one of the rows indicating that the pump status data for one of the patient rooms is “Infusing Near Complete;”

FIG. 5 is a screen shot of a third screen of the status board, similar to FIGS. 3 and 4, but showing one of the rows indicating that the pump status data for one of the patient rooms is “Infusing Complete;”

FIG. 6 is a screen shot of the wireless mobile device showing a pump status message of “Infusion Complete” and showing user inputs labeled “Accept,” “Accept & Call,” and “Re-Route;” and

FIG. 7 is a block diagram of a portion of the healthcare information system showing the pump gateway communicatively coupled to the enterprise gateway and to an EMR server and showing the EMR server also being communicatively coupled to the enterprise gateway.

### DETAILED DESCRIPTION

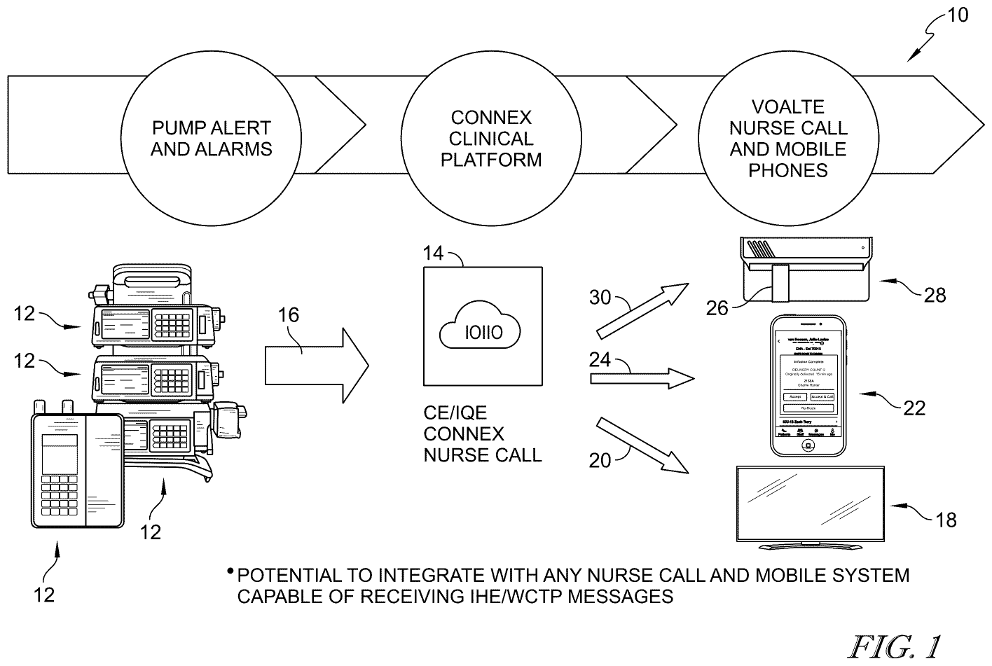

As shown in FIG. 1, a healthcare information system 10 includes multiple infusion pumps 12 that communicate alerts and alarms (these terms are used interchangeably herein) to a clinical platform 14 as indicated diagrammatically by arrow 16. These alerts and alarms communicated from infusion pumps 12 are considered herein to be included in the phrase pump status data. In the illustrative example, the clinical platform 14 is the CONNEX® clinical platform available from Baxter International Inc. of Deerfield, Illinois, U.S.A. The pump status data from infusion pumps 12 received at clinical platform 14 is eventually promulgated to a status board 18 of a nurse call system as indicated by arrow 20 and to a wireless mobile device 22, illustratively a mobile phone, as indicated by arrow 24, for display. The pump status data from infusion pumps 12 is also used by the nurse call system to illuminate a pump status section or zone 26 of a dome light 28 of the nurse call system as indicated by arrow 30. In the illustrative example, system 10 includes the VOALTE® nurse call system and mobile phones 22 run the VOALTER communication application, each of which is available from Hill-Rom Company, Inc. of Batesville, IN, U.S.A.

Although FIG. 1 illustrates a single wireless communication device 22, it should be appreciated that system 10 includes a plurality of such wireless communication devices 22, each of which is carried by a respective caregiver. Furthermore, it should be appreciated that a healthcare facility employing system 10 includes a plurality of patient rooms, each of which may include one or more infusion pumps 12, depending upon the clinical treatment needs of the respective patients, and each of which has a respective dome light 28 mounted in one or more hallways of the healthcare facility adjacent to doors of respective patient rooms of the healthcare facility.

System 10, therefore, has three distinct devices for communicating pump status information or data to caregivers and these are status board 18, wireless mobile devices 22, and dome lights 28. By communicating pump status data to these three distinct devices 18, 22, 28, caregivers are able to quickly determine which patient rooms any infusion pump alerts are originating from so that attention to each of the alerts can be given in an expeditious manner, if warranted.

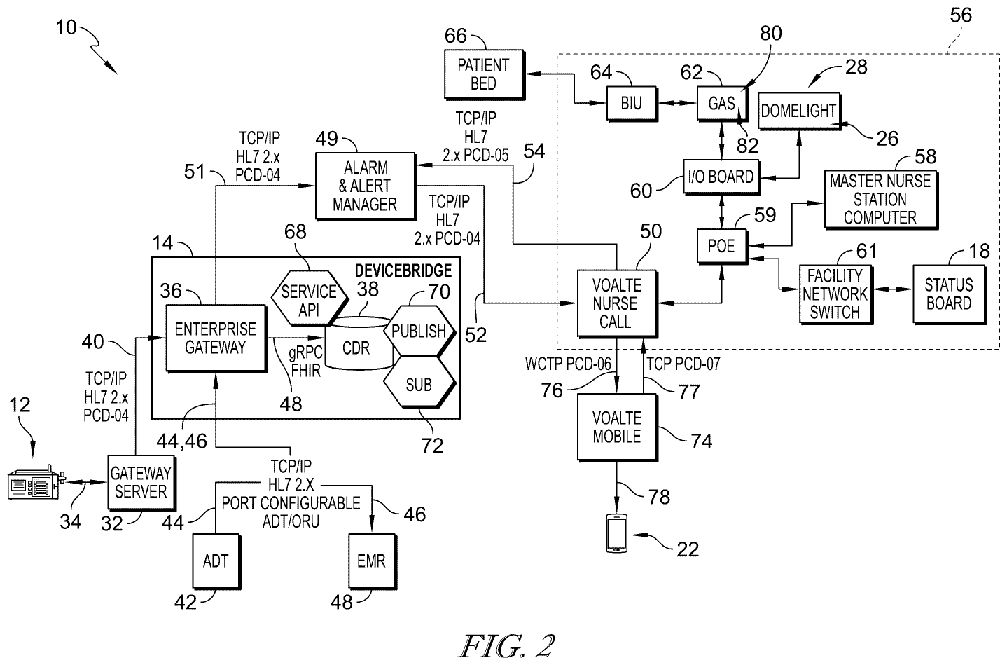

Referring now to FIG. 2, a more detailed illustration of system 10 is provided. System 10 has a pump gateway 32, which is a server in some embodiments, that is communicatively coupled to one or more pumps 12 as indicated by double headed arrow 34. In some embodiments of the healthcare information system 10, the pump status data is communicated from the one or more infusion pumps 12 to the infusion pump gateway 32 according to the IHE/HL7 protocol and/or according to any suitable wireless communication protocol. In the illustrative example, infusion pump 12 of FIG. 2 is of the type used in the Novum IQ Infusion Platform available from Baxter International Inc. of Deerfield, Illinois, U.S.A. Other types of infusion pumps 12, such as those provided in the Spectrum IQ Infusion System available from Baxter International Inc. of Deerfield, Illinois, U.S.A, are used in other implementations of system 10 in lieu of or in addition to the depicted Novum IQ infusion pump. Of course, different types of infusion pumps 12 from Baxter International Inc. and from other manufacturers may be used to treat different patients in different rooms of a healthcare facility. Thus, while only one infusion pump 12 is shown in FIG. 2, it should be understood that, in practice, system 10 has a multitude of infusion pumps 12 located throughout the healthcare facility.

System 10 interfaces or communicates with a network of the respective healthcare facility and such healthcare facility networks have wireless access points (WAP's) installed throughout the facility for wireless communication with various devices of all types using Wi-Fi signals. Thus, the present disclosure contemplates that infusion pumps 12 communicate wirelessly with one or more of such WAP's to send and receive wireless signals, including sending pump status data wirelessly. WAP's of healthcare facility also have wired connections, such as Ethernet cables, to the healthcare facility network and its remaining infrastructure. Accordingly, double headed arrow 34 in FIG. 2 is intended to represent the one or more WAP's, the wired connections, and the other network infrastructure that is employed to communicate pump status data from pumps 12 to pump gateway 32. In some embodiments of system 10, the wireless communications from pumps 12 to pump gateway 32 are formatted according to the Integrating the Healthcare Experience/Health Level Seven (IHE/HL7) protocol. Other protocols, however, may be employed if desired.

Clinical platform 14 includes an enterprise gateway 36 and a clinical data repository database 38 as shown in FIG. 2. In FIG. 2, the illustrative clinical platform 14 is a DeviceBridge (formerly, the Baxter Digital Health Platform (BDHP)) which is available from Baxter International Inc. of Deerfield, Illinois, U.S.A. In some embodiments of clinical platform 14, the DeviceBridge includes the CONNEX® clinical platform mentioned previously. Enterprise gateway 36 receives pump status data from pump gateway 32 over a communication link 40 such as one or more Ethernet cables and/or other components or other types of cables. In the illustrative example, the pump status data is transmitted from pump gateway 32 to enterprise gateway 36 according to the Transmission Control Protocol/Internet Protocol (TCP/IP), HL7 2.x, PCD-04 protocol.

In some embodiments, the pump status data transmitted over communication link 40 does not include any location data for the pump(s) 12 from which the pump status data originated. Thus, the present disclosure contemplates that, in some embodiments, enterprise gateway 36 queries an admission, discharge, and transfer (ADT) system or server 42 to obtain location data, or more accurately stated, to obtain patient-to-room association data. In the illustrative example of FIG. 2, ADT server 42 communicates the patient-to-room association data (e.g., location data) to enterprise gateway via a communication link 44. In some embodiments, pump-to-patient association data is entered at the one or more infusion pumps 12 or entered on an administration computer linked to gateway 32. In such embodiments, the messages communicated over communication link 40 include pump-to-patient association data. In other embodiments, pump-to-patient or pump-to-room association data is entered at an ADT computer linked to ADT server 42. In still other embodiments, pump-to-room association data is entered at infusion pump 12 and the messages communicated over communication link 40 includes the pump-to-room association data. Thus, the PCD-04 messages sent over communication link 40 may, or may not, include pump-to-room association data (i.e., location data for the infusion pump 12). If such data is not included in the messages sent over communication link 40, then enterprise gateway 32 communicates with ADT server 42 to obtain the pump-to-room association or location data.

The patient-to-room association data is transmitted from ADT server to enterprise gateway 36 over communication link 44 according to the TCP/IP, HL7 2.x protocol. In turn, enterprise gateway 36 transmits the pump status data, along with the corresponding patient-to-room association data, via communication link 46 to an electronic medical records (EMR) system or server 48. That is, the patient-to-room association data is appended to the pump status data for subsequent communications so that the pump status data can be associated with the corresponding patient and/or patient room by the EMR server 48. Communications between enterprise gateway 36 and EMR server 48 over communications link 46 are also according to the TCP/IP, HL7 2.x protocol. Furthermore, the communications over link 44 includes HL7 ADT messages and the communications over link 46 includes HL7 Observation Results (ORU) messages.

Still referring to FIG. 2, the pump status data, either alone or along with the appended patient-to-room association data, is transmitted to clinical data repository database 38 over a communication link 48. In the illustrative example, such data is transmitted over communication link 48 according to the open source Google Remote Procedure Calls (gRPC), Fast Healthcare Interoperability Resources 1 (FHIR) protocol. Additionally, the pump status data with the patient-to-room association data appended is transmitted from enterprise gateway 36 to an alarm manager 49 over a communication link 51. Alarm manager 49 communicates with a nurse call server 50 over a communication link 52 so that the pump status data can be associated with the corresponding patient and/or patient room by the nurse call server 50.

In the illustrative example, the pump status data with the corresponding patient-to-room association data appended is transmitted from enterprise gateway 36 the alarm manager 49 over communication link 51 according to the TCP/IP, HL7 2.x, PCD-04 protocol. The messages from the alarm manager 49 transmitted to nurse call server 50 over communication link 52 are also formatted according to the TCP/IP, HL7 2.x, PCD-04 protocol. Communications from nurse call server 50 to alarm manager 49 are transmitted over a communication link 54 and, in the illustrative embodiment, such communications are formatted according to the TCP/IP, HL7 2.x, PCD-05 protocol.

Alarm manager 49 is embodied as alarm manager software installed on an alarm manager server that is separate from enterprise gateway 36 and nurse call server 50 in the illustrative embodiment. In other embodiments, alarm manager 49 is embodied as alarm manager software installed on enterprise gateway 36 or another server of clinical platform 14 or nurse call server 50. Alarm manager 49 operates to manage medical device alarms, and in some embodiments, includes one or more of the following features: customizable alarm thresholds, prioritization of alarms, provision of signals providing visual and audible alerts on other devices, and data analysis to identify and minimize nuisance alarms. In some embodiments, alarm manager 49 receives alarm messages from cardiac monitors that may have adjustable heart rate alarm limits, ventilator alarms for low tidal volume, infusion pump alarms for occlusion or other infusion pump alarm or alert condition, blood pressure monitors that may have customizable thresholds, and other smart alarm systems that monitor patient data and/or patient care device data that generate alerts or alarms. The terms “alarm” and “alert” are used interchangeably herein.

Nurse call server 50 is included as one of the components of a nurse call system 56 as shown in FIG. 2. In addition to nurse call server 50, nurse call system 56 includes a master nurse station computer 58 that is communicatively coupled to nurse call server 50 via a Power over Ethernet (POE) switch 59. In the illustrative example, nurse call server 50 is also coupled to status board 18 via POE switch 59 and a facility network switch 61. Status board 18 is also communicatively coupled via switches 59, 61 to master nurse station computer 58 and an input/output (I/O) board 60 that is packaged as a unit with dome light 28. As noted above, a unit of a healthcare facility has dome lights 28, and therefore, I/O boards 60, for each patient room of the unit, but only dome light 28 and one I/O board 60 is shown in FIG. 2 for ease of illustration. Nurse call system 56 further includes a graphical audio station (GAS) 62 (sometimes referred to as a graphical room station (GRS)) in each room that is communicatively coupled to the respective I/O board 60. Nurse call system 56 further includes a bed interface unit 64 in each room that is communicatively coupled to the respective I/O board and that is communicatively coupled to a patient bed 66 in the respective room.

Bed status data from beds 66 in the various patient rooms of a unit of a healthcare facility are communicated via respective BIU's 64, I/O boards 60, and POE switch 59 to master nurse station computer 58 and to nurse call server 50. Nurse call server 50, in turn, transmits the bed status data via communication link 54, alarm manager 59, and communication link 51 to enterprise server 36 which forwards the bed status data to clinical data repository database 38 via communication link 48. As shown in FIG. 2, clinical data repository database 38 has a service application interface (API) 68 to provide pump status data, bed status data, location data, etc. to a remote service system (not shown). Clinical data repository database 38 also has a publish application 70 and a subscribe application 72 to provide pump status data, bed status data, location data, etc. to third parties that are interested in receiving such data.

Still referring to FIG. 2, illustrative system 10 includes a communications server 74 that is communicatively coupled to nurse call server 50 via a first communication link 76 and a second communication link 77. Any pump status data, bed status data, and location data transmitted over communication link 76 from server 50 to server 74 is formatted according to the WCTP, PCD-06 protocol in some embodiments, or according to the HL7, PCD-06 protocol in other embodiments. In the illustrative examples, any transmissions from server 74 to server 50 over communication link 77 are formatted according to the TCP, PCD-07 protocol. Communications server 74 initiates the transmission of the pump status data, and other data such as bed status data and/or location data, to wireless communication devices 22 of respective caregivers via a wireless communication link 78 using the Wireless Communications Transfer Protocol (WCTP) in some embodiments. It should be appreciated that other telecommunications infrastructure, including wireless access points, routers, digital branch exchanges (DXP's), and the like, is involved in delivering messages from communications server 74 to wireless communication devices 22 in some embodiments.

The present disclosure contemplates that wireless communication devices 22 are operable to open up an audio channel to graphical audio stations 62 in the various patient rooms. In this regard, graphical audio stations 62 each include a speaker 80 and a microphone 82 that enable a caregiver to converse with the patient in the respective patient room. Caregivers may choose to open up such an audio channel based on alert messages pertaining to the pump status data, bed status data, nurse calls sent by the patient, etc.

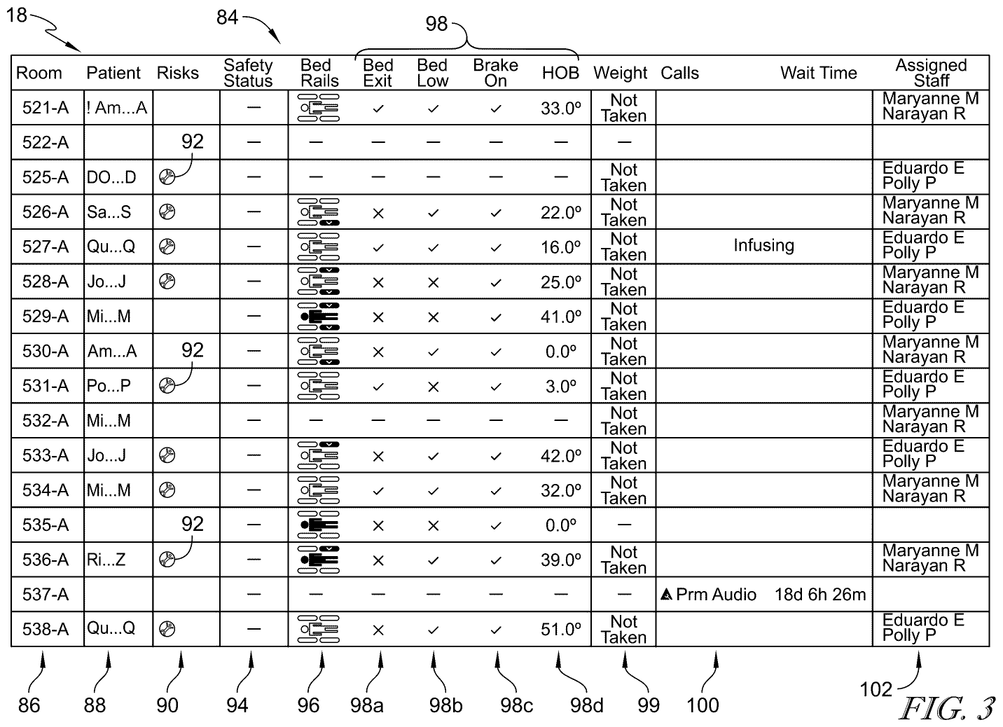

Referring now to FIG. 3, an example of a screen shot of a first screen 84 of status board 18 of nurse call system 56 is shown. Status board 18 is located at a master nurse call station of a unit of the healthcare facility. The status board 18 is communicatively coupled to nurse call server 50 and master nurse station computer 58, as noted above, and displays in a table a wide variety of alert and device status data that originate from patient rooms of the healthcare facility. It should be appreciated that status board 18 is a large display screen on the order of about 3 feet to about 6 feet in diagonal, or more, and that is typically mounted at the master nurse station on a wall so as to be viewable by all staff members that are in close proximity to the master nurse station.

Going from left to right in FIG. 3, screen shot 84 includes a room column 86 which shows the room numbers of the various rooms in the unit associated with the status board 18 and a patient column 88 showing, in a Health Insurance Portability and Accountability Act (HIPAA) compliant format, the names of the patients in the various rooms. The HIPAA compliant format is, for each patient, the first two letters of the patient's last name, a set of ellipses, and the first letter of the patient's first name (e.g., “Ab. . . . C”).

The next column in FIG. 3 is a risks column 90 which has icons to indicate a corresponding patient's medical risks. Up to three icons can be shown in column 90 for each patient in the illustrative embodiment. In the illustrative example, a falls risk icon 92 is shown in various rows to indicate that the corresponding patient is a falls risk. Examples of other risk icons (not shown) are a pulmonary risk icon (e.g., a lungs icon) to indicate that the corresponding patient is at risk of developing pulmonary complications such as ventilated assisted pneumonia (VAP), and a skin risk icon (e.g., a bandage icon) to indicate that the corresponding patient is at risk of developing pressure ulcers (aka bed sores or decubitus ulcers). To the right of column 90 on screen 84 is a safety status column 94 that, under appropriate circumstances, shows icons indicative of whether a safety protocol is being followed (e.g., icon is a green circle) or is being violated (e.g., icon is a red triangle) or that the patient is out of the room (e.g., a blue door and person icon). In the illustrative FIG. 3 example, however, each row of column 94 has a dash to indicate that safety status is not being monitored.

The next column of screen shot 84 of status board 18 is a bed rails column 96 that shows a patient and bed siderails icon with color coding to indicate the status of each of the siderails of each patient bed 66 in each of the corresponding patient rooms. The icon is a simplified view from above the patient such that four line segments around the patient icon portion are representative of the four bed siderails. The patient icon portion has a small circle indicative of the patient's head such that the two line segments adjacent the small circle of the patient icon portion represent the head end siderails of the corresponding patient bed and the other two line segments represent the foot end siderails of the corresponding patient bed.

With regard to the color coding of the icons appearing in column 96 for siderail status, green indicates that the corresponding siderail is in a raised position inhibiting patient exit from the respective bed 66; yellow indicates that the corresponding siderail is lowered but does not result in an alert or alarm condition, such as if a caregiver is in the patient room and has assisted the patient in getting out of bed 66 or the PPM/bed exit system is not armed such that patient position and bed exit is not being monitored, or if no patient is assigned to the corresponding bed; and red indicates that the corresponding siderail is lowered and results in an alarm condition which, in turn, results in a call being placed to one or more caregivers assigned to the respective patient or patient room. The patient portion of the icons appearing in column 96 is also color coded such that green indicates that a patient is sensed to be in the corresponding patient bed 66 and gray indicates that no patient is sensed to be in the corresponding patient bed 66.

To the right of column 96 in screen shot 84 is a bed status column 98 which includes four sub-portions including sub-portion 98a to indicate bed exit status for each patient bed in the corresponding unit, sub-portion 98b to indicate bed low status for each patient bed in the corresponding unit, sub-portion 98c to indicate brake on status for each patient bed in the corresponding unit, and sub-portion 98d that contains an angle at which a head section of the corresponding bed 66 is raised relative to the upper frame of the corresponding bed or relative to horizontal depending upon the type of angle sensor used (e.g., potentiometer or accelerometer).

In sub-portion 98a of column 98 a check mark means the PPM/bed exit system of bed 66 is armed; an X mark means that the PPM/bed exit system is not armed; an empty triangle means the PPM/bed exit system it not armed but should be for a falls risk patient, or that a patient is absent from the room or not assigned to the room; a dash indicates that no patient bed 66 is currently transmitting bed status data from the corresponding room; and a filled in triangle means that the PPM/bed exit system is armed and is alarming because the corresponding patient has exited the bed or has moved toward exiting the bed by a threshold amount (e.g., depending the mode of the PPM/bed exit system).

With regard to sub-portion 98b of column 98, a check mark means that an upper frame of the corresponding bed 66 is in its low position, an X mark means the upper frame of the corresponding bed 66 is not in its low position, and a dash indicates that no patient bed 66 is currently transmitting bed status data from the corresponding room. With regard to sub-portion 98c of column 98, a check mark means that caster brakes of the corresponding bed are braked (e.g., locked or “on”), an X mark means that the caster brakes of the corresponding bed are unbraked (e.g., unlocked or “off”), an empty triangle means that a bed brakes alert is occurring such as for a corresponding falls risk patient, and a dash indicates that no patient bed is currently transmitting bed status data from the corresponding room. With regard to sub-portion 98d of column 98, some patient beds permit a HOB alarm to be turned on which results in an alarm being sent if the HOB angle drops below a threshold angle, such as 30 or 45 degrees, for example.

To the right of column 98 on screen 84 is a weight column 99 that displays a weight of a corresponding patient if the patient's weight has been taken using a weigh scale system of the respective patient bed 66. The patient weight is shown in pounds (lbs.) or kilograms (kgs.) at the discretion of the user. In the illustrative FIG. 3 example, the text “Not Taken” is shown for each of the patients to indicate that the patient weight has not been taken at each of the respective patient beds 66.

The next column on screen 84 of status board 18 is a calls column 100 which shows all of the active calls and/or notifications for the various patient rooms and, in some embodiments, also shows the wait time, in minutes (or, alternatively, minutes and seconds), subsequent to the occurrence of the respective call. Such calls in column 158 are based on alerts or alarms from equipment in the room, such as the infusion pumps 12 and patient beds 66, or based on a patient pressing a nurse call button on the corresponding patient bed 66 or a pillow speaker unit or based on a caregiver or other staff action in the patient room. For example, if a caregiver has pulled a code blue lever on a room station (e.g., GAS 62 or a standard room station) in the corresponding patient room, then a “+Code Blue” message appears in column 100. The term “code blue” means that the corresponding patient has gone into respiratory or cardiac arrest and likely requires cardiopulmonary resuscitation (CPR).

Other calls that may appearing in column 100 depending upon corresponding circumstances include the following: “Bed Disconnected Call” to indicate that the patient bed 66 in the corresponding room has become unplugged from power or from the nurse call system 12; “Too Few Rails Up” to indicate that one or more siderails of the patient bed 66 have been lowered for a patient who is a falls risk, for example; “Bed Exit Off Alert” to indicate that the PPM/bed exit system of the patient bed 66 in the room 66 of a patient who is a falls risk has been disarmed (or turned off so as not to monitor the patient's position or bed exit); “Normal” to indicate that the patient has pressed a nurse call button to speak with or summon a caregiver; “Staff Call” to indicate that a staff member in a particular patient room is placing a call to another staff member using the corresponding room station 62, a badge or tag with communication capability, or the associated mobile phone 22; “Staff Emergency” to indicate that one staff member has placed an emergency call using a staff locating tag/badge, or using a room station to summon one or more other staff members, or using the associated mobile phone 22 to summon one or more other staff members; “Bed Exit Alarm Call” to indicate the corresponding patient has gotten out of bed 66 when the patient PPM/bed exit system of the associated patient bed has been armed (e.g., turned on) to monitor for patient bed exit or movement toward bed exit; and “Bed Brakes Alert” to indicate that caster brakes of the corresponding patient bed 66 have been unbraked or released.

The foregoing examples of calls that may appear in column 100 are not intended to be exhaustive and other call messages are displayed in column 100 as appropriate at the discretion of a designer and/or administrator of system 10. However, for purposes of the present disclosure, messages (aka notifications) pertaining to the pump status data originating from infusion pumps 12 in the various patient rooms are of interest. In particular, column 100 of screen 84 in FIG. 3 indicates that one or more infusion pumps 12 in the row corresponding to room 527-A is/are “Infusing” the respective patient. That is, the one or more infusion pumps 12 in room 527-A are operating to treat the respective patient by infusing one or more fluids into the circulatory system of the patient. In this regard column 100 of status board is considered to serve as a pump status column to display alert/alarm/status notifications corresponding to the pump status data of an associated infusion pump 12.

Continuing on from left to right in FIG. 3, an assigned staff column 102 shows the staff members who are assigned to each of the patient rooms in the unit. In the illustrative example, there are two staff members (e.g., caregivers) assigned to each patient room having a patient. The format for showing the assigned caregivers in column 102 is full first name followed by first letter of last name. In some embodiments, a role designator, typically an acronym, for each staff member is shown to the right of each of the staff member names. For example, the role designators include “RN” for registered nurse and “CNA” for certified nursing assistant. In addition, the phone numbers or extensions for each staff member, which may include the phone numbers of the associated mobile phones 22, may be provided in column 102 in some embodiments.

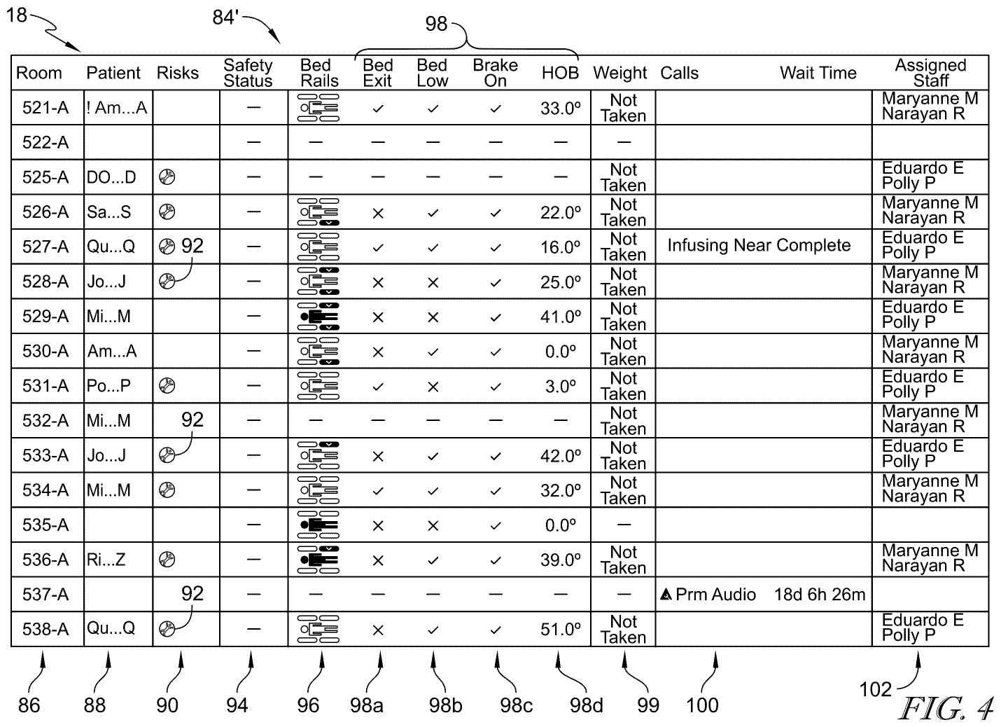

Referring now to FIG. 4, a screen shot of a second screen 84′ of the status board, similar to screen 84 of FIG. 3, but showing column 100 displaying that the pump status data for the one or more infusion pumps 12 in room 526-A is indicating, “Infusing Near Complete.” According to the present disclosure, infusion is considered to be “Near Complete” if the one or more infusions pumps 12 are within thirty minutes or less, such as five minutes just to give one arbitrary time threshold example, of infusing an associated patient. Time thresholds that are greater than thirty minutes or less than thirty minutes for considering infusion to be “Near Complete,” are within the scope of the present disclosure at the option of the designer of system 10. In FIG. 4, portions of screen 84′ that are the same, or substantially the same, as portions of screen 84 are denoted by like reference numbers and the descriptions are not repeated.

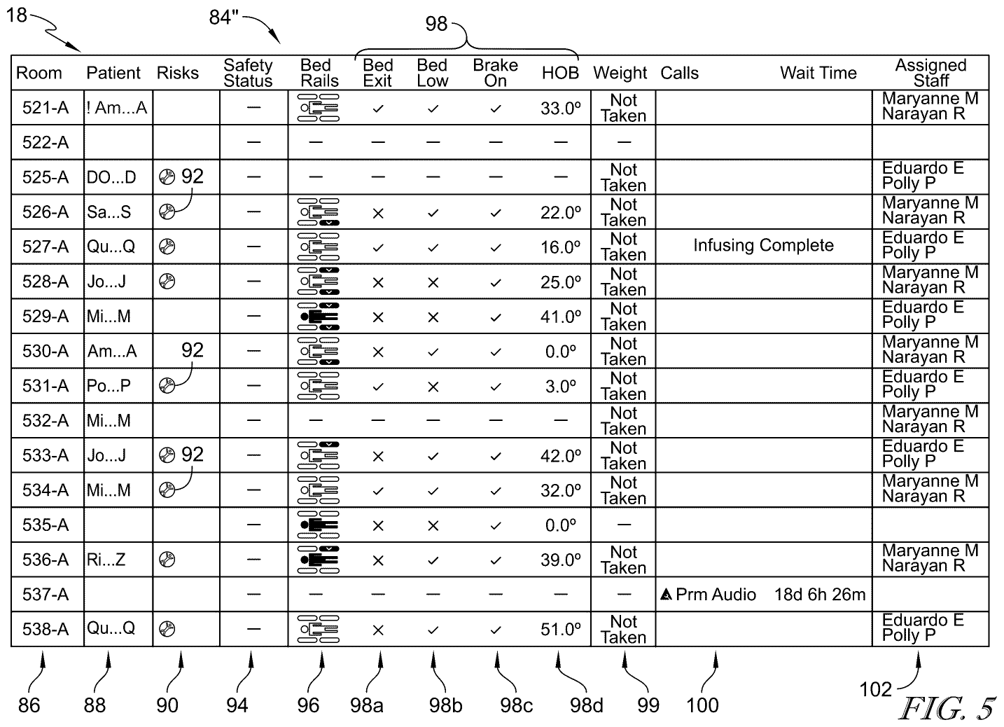

Referring now to FIG. 5, a screen shot of a third screen 84″ of the status board, similar to screens 84, 84′ of FIGS. 3 and 4, respectively, but showing column 100 displaying that the pump status data for the one or more infusion pumps 12 in room 527-A is indicating, “Infusing Complete.” That is, infusion is considered to be “Complete” after the one or more infusion pumps 12 have finished infusing an associated patient. In FIG. 5, portions of screen 84″ that are the same, or substantially the same, as portions of screens 84, 84′ are denoted by like reference numbers and the descriptions are not repeated.

In the illustrative example of system 10, the messages displayed on status board 18 based on pump status data include notifications stating, “Infusing,” “Infusing Near Complete,” and “Infusing Complete.” It should be appreciated that other messages of similar import (i.e., equivalent messages) may be used in lieu of the given examples. For example, “Infusion in Process” may be used in lieu of “Infusing;” “Infusing Approaching Completion” may be used in lieu of “Infusing Near Complete;” and “Infusion Finished” may be used in lieu of “Infusing Complete;” just to provide a few equivalent messages. Another example of a message that may appear in column 100 is “Fluid Bag Near Empty” in the case of infusion involving fluid bags. In addition, error messages or alarm messages based on the pump status data may be provided in column 100, as appropriate, based on the pump status data. Such error messages or alarm messages may include messages pertaining to infusion pump motor faults (e.g., over temperature), infusion pump circuit errors (e.g., power disconnect or low battery), fluid line blockage or kinking (aka occlusion), and mechanical faults (e.g., door latch errors).

With continued reference to FIGS. 3-5, pump status sections 26 of dome lights 28 are illuminated in different manners depending upon the messages in column 100 based on the pump status data. In particular, the pump status section 26 of the respective dome light 28 is illuminated continuously in response to the pump status data indicating that one or more infusion pumps 12 are infusing an associated patient, in which case “Infusing” appears in column 100 in the row corresponding to the respective room. The pump status section 26 of the respective dome light 28 is illuminated so as to flash at a slow rate in response to the pump status data indicating that the corresponding one or more infusion pumps 12 are nearing completion by being within thirty minutes or less, such as about five minutes just to give one arbitrary example, of infusing an associated patient, in which case “Infusing Near Complete” appears in column 100 in the row corresponding to the respective room. In this regard, flashing at a slow rate is contemplated by the present disclosure as flashing at most once per second just to give an arbitrary example of a slow flash rate. It should be appreciated that time thresholds for nearing completion of infusion that are greater than thirty minutes (e.g., 45 minutes, one hour, etc.) are within the scope of the present disclosure.

The pump status section 26 of the respective dome light 28 is illuminated so as to flash at a fast rate in response to the pump status data indicating that the respective one or more infusion pumps 12 have completed infusing the associated patient, in which case “Infusing Complete” appears in column 100 in the row corresponding to the respective patient room. In this regard flashing at a fast rate is contemplated by the present disclosure as flashing at least twice per second just to give one arbitrary example of a fast flash rate. It should be appreciated that slow flash rates slower than once per second (e.g., once every 1.5 seconds, once every 2 seconds, etc.) and fast flash rates greater than twice per second (e.g., 3 times per second, 4 times per second, etc.) are within the scope of the present disclosure.

In some embodiments, the pump status sections 26 of the dome lights are magenta in color when illuminated, which is a color that is different than the color at which all other sections or zones of dome lights 28 are illuminated. The other zones may be illuminated so as to be white, yellow (aka amber), green, or red, for example. By illuminating pump status zone 26 of dome lights 28 in a different color than the other zones, the infusion status of a patient in the corresponding room stands out to the caregivers viewing the dome lights 26 and the flashing of zones 26 makes it easier for caregivers to discern which patient rooms have patients that require attention, or will soon require attention, concerning the corresponding infusion pump(s) 12. Thus, the pump status section 26 of each dome light 28 is illuminated in a dedicated color for pump status, such that the magenta color is just given as one arbitrary color example.

The present disclosure further contemplates that the color of the pump status section 26 is configurable in some embodiments, such as being customer configurable to match a color of a housing of the particular type of infusion pump that is used predominantly throughout a healthcare facility or within a particular unit or wing of the healthcare facility. In this regard, dome lights 28 having multi-color LED's that are programmable to produce a desired color are within the scope of the present disclosure. To achieve the multi-color illumination, a set of LED's that each produce a dedicated color (e.g., red, blue, green, yellow, etc.) are provided in each zone of the dome light 28 in some embodiments and then the on/off state of each LED and/or the intensity or brightness of illumination of each LED is then controlled to produce the desired color. For example, the magenta color mentioned herein can be produced by turning on a red LED and a blue LED at the same time and with the same intensity/brightness. A lens or other light transmissive element (e.g., acrylic light pipe) serves to combine the colors of the individual LED's together to provide the desired overall color of illumination of the corresponding zone, such as zone 26, of the dome light 28.

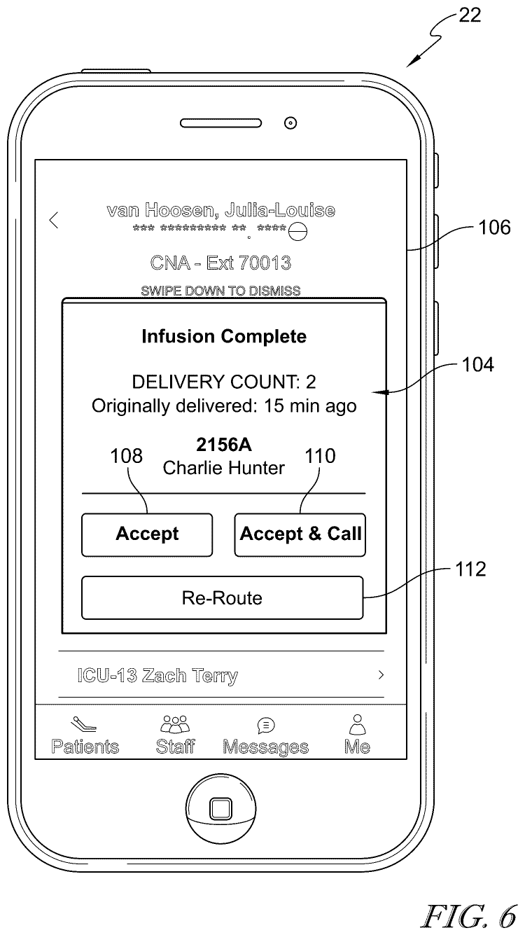

Referring now to FIG. 6, an example of a screen shot of a pump status window 104 that appears on a display screen of a caregiver's wireless mobile device 22 in response to completion of infusion for a patient in a particular room. Thus, window 104 is shown on mobile wireless device 22 generally at the same time that “Infusing Complete” appears in column 100 of status board 18 and generally at the same time that pump status section 26 of dome light 28 begins to flash at the fast rate for the associated patient room. In the illustrative example, a pump status message of “Infusion Complete” is shown at the top of window 104. Beneath the pump status message in window 104 is the text “DELIVERY COUNT:2” to indicate that the corresponding patient has undergone two infusion treatments. Of course, the number next to “DELIVERY COUNT:” varies depending upon the number of infusion treatments for the associated patient.

Still referring to FIG. 6, the text “Originally delivered: 15 minutes ago” appears in window 104 of wireless mobile device 22 beneath the text “DELIVERY COUNT:2” to indicate an amount of time that has elapsed subsequent to the infusion treatment being originally delivered. Thus, the time since original delivery of the infusion treatment is updated in window 104 on a minute-by-minute basis in some embodiments. A room number in which the one or more infusion pumps 12 having the pump status data causing window 104 to appear on display screen 106 of mobile phone 22 is shown beneath the text “Originally delivered: 15 minutes ago.” In the illustrative example of FIG. 6, the room number is “2156A.” Beneath the room number is the first and last name of the patient assigned to room. In the illustrative example, the patient's name is “Charlie Hunter.” Because the mobile phone 22 is not publicly viewable but is generally only viewable by the caregiver transporting the mobile phone 22, the patient's name does not need to be shown in a HIPAA-compliant format.

With continued reference to FIG. 6, three user input buttons 108, 110, 112 are provided in window 104 beneath the patients name. The three buttons 108, 110, 112 are labeled “Accept,” “Accept & Call,” and “Re-Route;” respectively. Selection of Accept button 108 by the caregiver indicates to nurse call system 56 that the caregiver accepts responsibility for responding to the pump status alert (e.g., “Infusion Complete” in the illustrative example). Selection of Accept & Call button 110 by the caregiver indicates to nurse call system 56 that the caregiver accepts responsibility for responding to the pump status alert and also opens an audio channel to the GAS 62 in the corresponding patient room (e.g., room 2156A in the illustrative example) so that the caregiver can speak with the patient. For example, the caregiver may tell the patient that the caregiver is on their way to remove the infusion line from the patient's arm.

Selection of Re-Route button 112 in window 104 indicates to the nurse call system 56 that the pump status alert should be re-routed (e.g., escalated) to another caregiver, such as a secondary or back-up caregiver, for response. Thus, in response to selection of Re-Route button 112, nurse call system 56 initiates a communication to the wireless mobile device 22 of the secondary caregiver to cause window 104 to appear on the display screen 106 of the secondary caregiver's wireless mobile device 22. It should be appreciated, therefore, that display screens 106 of wireless mobile devices 22 are touchscreen displays. It should be appreciated that the notification message appearing on mobile device 22 corresponds to the notification appearing on status board 18. Thus, each of the notification message examples given above pertaining to infusion pump 12 that may appear on status board 18, also appear on the mobile device of one or more assigned caregivers in lieu of the depicted example “Infusion Complete” of FIG. 6.

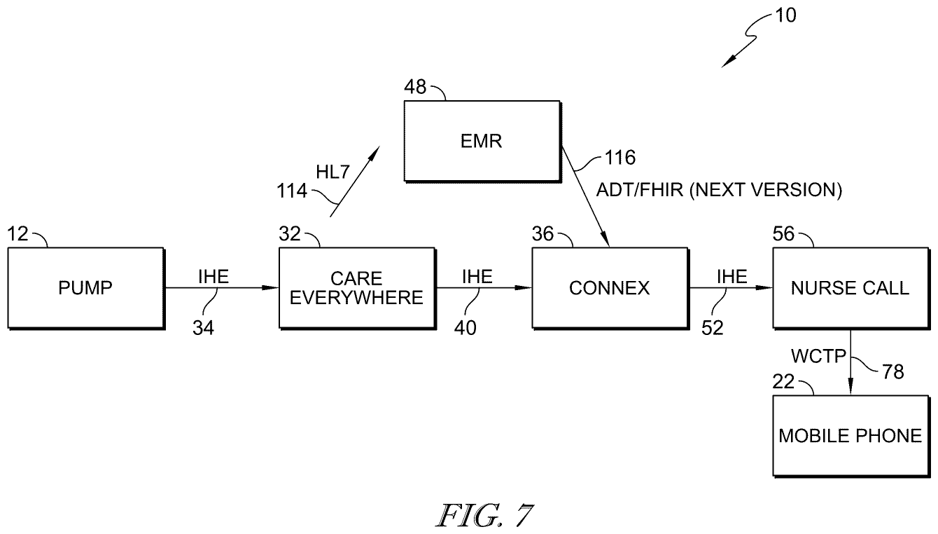

Referring now to FIG. 7, a block diagram of a portion of the healthcare information system 10 is shown in which pump gateway 32 is communicatively coupled to the enterprise gateway 36 via communication link 40, as discussed above, but is also communicatively coupled to EMR server 48 via a communication link 114. This is an alternative communication arrangement as compared to example of system 10 shown in FIG. 2. In the illustrative FIG. 7 example, the pump status messages from pump gateway 32 to EMR server 48 are formatted according to the HL7 protocol. Also in the FIG. 7 example, pump gateway 32 still sends pump status data to enterprise server 36 via communication link 40. However, EMR server 48 is communicatively coupled to the enterprise gateway 36 via a communication link 116. The messages sent from EMR server 48 to enterprise server 36 via communication link 116 are formatted according to the Fast Healthcare Interoperability Resources 1 (FHIR) protocol. ADT data is also included in the messages communicated via communication link 116 in the illustrative FIG. 7 example. The remaining blocks and communications links in FIG. 7 have been discussed above and so the descriptions are not repeated.

Additional details of nurse call system 56 and alternative nurse call systems, as well as components thereof, are shown and described in U.S. Pat. Nos. 8,598,995; 8,384,526; 8,169,304; 8,046,625; 7,746,218; 7,538,659; 7,319,386; 7,242,308; 6,897,780; 6,362,725; 6,147,592; 5,838,223; 5,699,038 and 5,561,412, all of which are hereby incorporated by reference herein in their entirety to the extent that they are not inconsistent with the present disclosure which shall control as to any inconsistencies. It should be noted that U.S. Pat. No. 8,384,526 includes additional details pertinent to dome light 28 of the present disclosure. Dome light 28 is referred to as an “indicator apparatus” in U.S. Pat. No. 8,384,526.

In some embodiments, bed interface unit (BIU) 64 is referred to as a network interface unit (NIU), and examples of such interface units are shown and described in U.S. Pat. Nos. 7,538,659 and 7,319,386 and in U.S. Patent Application Publication Nos. 2009/0217080, 2009/0212925 and 2009/0212926, each of which is hereby expressly incorporated by reference herein in its entirety to the extent that they are not inconsistent with the present disclosure which shall control as to any inconsistencies. In other embodiments, bed interface unit 64 communicates wirelessly with bed 66 as shown and described in U.S. Patent Application Publication No. 2022/0233382 which is hereby incorporated by reference herein in its entirety to the extent that it is not inconsistent with the present disclosure which shall control as to any inconsistencies.

When terms of degree such as “generally,” “substantially,” and “about” are used herein in connection with a numerical value or a qualitative term susceptible to a numerical measurement, it is contemplated that an amount that is plus or minus 10 percent, and possibly up to plus or minus 20 percent, of the numerical value, is covered by such language, unless specifically noted otherwise, to at least account for manufacturing tolerances. Otherwise, a suitable definition for “generally,” “substantially,” and “about” is largely, but not necessarily wholly, the term specified.

When the terms “a” or “an” or the phrases “one or more” or “at least one” are used herein, including in the claims, they are all intended to be synonymous and mean that one or more than one of the thing recited may be present. Similarly, when the phrases “a plurality” or “two or more” or “at least two” or “a pair” are used, they are all intended to be synonymous and mean that two or more than two of the thing recited may be present.

Although certain illustrative embodiments have been described in detail above, variations and modifications exist within the scope and spirit of this disclosure as described and as defined in the following claims.

1. A healthcare information system for use in a healthcare facility having a network including wireless access points, the healthcare information system comprising
an infusion pump gateway,
one or more infusion pumps configured to transmit pump status data wirelessly to at least one of the wireless access points of the healthcare facility for forwarding to the infusion pump gateway,
an enterprise gateway communicatively coupled to the infusion pump gateway and configured to receive the pump status data from the infusion pump gateway,
a plurality of wireless mobile devices each carried by a respective caregiver of the healthcare facility, and
a nurse call system including a nurse call server, a status board, and a plurality of dome lights mounted in one or more hallways of the healthcare facility adjacent to doors of respective patient rooms of the healthcare facility, the nurse call server being communicatively coupled to the enterprise gateway and configured to receive the pump status data from the enterprise gateway, the nurse call server being configured to promulgate the pump status data for display on the status board and for display on at least one of the plurality of wireless mobile devices, the nurse call server also being configured to cause a pump status section of at least one of the plurality dome lights to be illuminated in a manner that is dependent upon the content of the pump status data.

2. The healthcare information system of claim 1, wherein the nurse call system includes a master nurse station computer communicatively coupled to the nurse call server and communicatively coupled to the status board, the pump status data also being sent to the master nurse station computer which displays of the pump status data.

3. The healthcare information system of claim 1, wherein the pump status data is displayed on the status board in a row of a table and wherein the row also displays any one or more of the following: a room number of the respective room in which the at least one infusion pump is located, a patient name in HIPAA-compliant format of a patient assigned to the respective room, patient risk information, bed status information of a patient bed in the respective room, and names of caregivers assigned to the patient or to the patient room.

4. The healthcare information system of claim 1, wherein the nurse call system includes a graphical audio station in the respective room in which the one or more infusion pumps are located, the master nurse station computer being communicatively coupled to the graphical audio station, and the plurality of wireless mobile devices are configured to display a user input which is displayed along with the pump status data and which is selectable to open an audio channel between the wireless mobile device and the graphical room station.

5. The healthcare information system of claim 1, wherein the pump status data is indicative any one or more of the following: infusing, infusing near complete, and infusing complete.

6. The healthcare information system of claim 1, wherein each of the plurality of wireless mobile devices is configured to display, along with the pump status data, any one or more of the following: a room number of the respective room in which the at least one infusion pump is located, a name of a patient in the respective room, a first user input to accept an alert associated with the pump status data, a second user input to place an audio call to the respective room, and a third user input to re-route the alert associated with the pump status data to another caregiver.

7. The healthcare information system of claim 6, wherein each of the plurality of wireless mobile devices is configured so that selection of the second user input also accepts the alert associated with the pump status data.

8. The healthcare information system of claim 1, wherein the pump status section of the at least one dome light is illuminated continuously in response to the pump status data indicating that the one or more infusion pumps are infusing an associated patient.

9. The healthcare information system of claim 1, wherein the pump status section of the at least one dome light is illuminated so as to flash at a slow rate of at most once per second in response to the pump status data indicating that the one or more infusion pumps are nearing completion by being within thirty minutes or less of infusing an associated patient.

10. The healthcare information system of claim 1, wherein the pump status section of the at least one dome light is illuminated so as to flash at a fast rate of at least twice per second in response to the pump status data indicating that the one or more infusion pumps have completed infusing the associated patient.

11. The healthcare information system of claim 1, wherein the pump status section of the at least one dome light is magenta in color when illuminated.

12. The healthcare information system of claim 1, wherein an alarm occurring at the one or more infusion pumps in a patient room results in the nurse call system displaying an alarm message on the status board and on one or more of the wireless mobile devices of caregivers assigned to the patient room and wherein acknowledgment of the alarm at the one or more infusion pumps, or rectification of a condition causing the alarm at the one or more infusion pumps, clears the alarm from the nurse call system which results in the alarm message no longer being displayed on the status board and no longer being displayed on the one or more of the wireless mobile devices of caregivers assigned to the patient room.

13. The healthcare information system of claim 1, wherein the enterprise gateway is communicatively coupled to an ADT server of the healthcare facility and receives patient-to-room association data from the ADT server.

14. The healthcare information system of claim 1, wherein the enterprise gateway is communicatively coupled to an EMR server of the healthcare facility and is configured to route the pump status data to the EMR server.

15. The healthcare information system of claim 1, further comprising a clinical data repository database and wherein the enterprise gateway is configured to route the pump status data to the clinical data repository database.

16. The healthcare information system of claim 1, wherein the pump status data includes a notification that a fluid bag is near empty.

17. The healthcare information system of claim 1, wherein the pump status data is communicated from the one or more infusion pumps to the infusion pump gateway according to the IHE/HL7 protocol.

18. The healthcare information system of claim 1, wherein the pump status data is communicated from the infusion pump gateway to the enterprise gateway according to the TCP/IP, HL7 2.x, PCD-04 protocol.

19. The healthcare information system of claim 1, wherein communications from the enterprise gateway to the nurse call system are according to the TCP/IP, HL7 2.x, PCD-04 protocol and wherein communications from the nurse call system to the enterprise gateway are according to the TCP/IP, HL7 2.x, PCD-05 protocol.

20. The healthcare information system of claim 1, further comprising a communications server communicatively coupled to the nurse call server and configured to receive the pump status data for forwarding to at least one wireless mobile device of the plurality of wireless mobile devices, and wherein communications from the nurse call server to the communications server are according to the HL7 PCD-06 protocol.

21. The healthcare information system of claim 20, wherein communications between the communications server and the at least one wireless mobile device of the plurality of wireless mobile devices are according to the Wireless Communications Transfer Protocol (WCTP).

22. The healthcare information system of claim 1, wherein the infusion pump gateway and the enterprise gateway are communicatively coupled to an EMR server of the network of the healthcare facility, wherein the pump status data is communicated from the infusion pump gateway to the EMR server according to the HL7 protocol, and wherein the pump status data is communicated from the EMR server to the enterprise gateway according to the Fast Healthcare Interoperability Resources 1 (FHIR) protocol.

23. The healthcare information system of claim 1, wherein the enterprise gateway is communicatively coupled to an ADT server of the network of the healthcare facility, wherein the enterprise gateway is configured to query the ADT server for patient-to-room association data, and wherein, in response to receiving pump status data from the one or more infusion pumps, the enterprise gateway is configured to append the patient-to-room association data to the pump status data for the patient treated by the corresponding one or more infusion pumps.

24. The healthcare information system of any of claim 1, wherein the nurse call server is communicatively coupled to the enterprise gateway via an alarm manager.
